SEQUENCE LISTING (40 sequences)

SEQ ID NO: 1 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtacgactgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatcagccaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 3 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 4 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 5 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 6 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccttcaaggtgctggacctgaagaactacatcgacaagcagctgctgcccatcctcaacaagcagagctgcagcccaagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 7 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSPSNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 8 - DNA, 1725 bp, synthetic construct; identical to SEQ ID NO: 6
  source 1..1725: mol_type = other DNA

SEQ ID NO: 9 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDACISQVNEKINQSLAFIRKSDELLHNVNAGKSTTNIMITTIIIVIIVILLSLIAVGLLLYCKARSTPVTLSKDQLSGINNIAFSN

SEQ ID NO: 10 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgctgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgcctgcatcagccaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 11 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDACISQVNEKINQSLAFIRKSDELLHNVNAGKSTTNIMITTIIIVIIVILLSLIAVGLLLYCKARSTPVTLSKDQLSGINNIAFSN

SEQ ID NO: 12 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgacctttaaggtgctggacctgaagaactacatcgacaagcagctgctgcccatcctgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgctgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgcctgcatcagccaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 13 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSPSNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDACISQVNEKINQSLAFIRKSDELLHNVNAGKSTTNIMITTIIIVIIVILLSLIAVGLLLYCKARSTPVTLSKDQLSGINNIAFSN

SEQ ID NO: 14 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgacctttaaggtgctggacctgaagaactacatcgacaagcagctgctgcccatcctgaacaagcagagctgcagccccagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgctgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgcctgcatcagccaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 15 - protein, 574 aa, synthetic construct
MELLILKANAITTILTAVTFCFASGQNITEEFYQSTCSAVSKGYLSALRTGWYTSVITIELSNIKKNKCNGTDAKVKLIMQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 16 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcatgcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 17 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 18 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 19 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYYNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 20 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtactacaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 21 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVYTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 22 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacaagaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtgtataccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 23 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSPSNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 24 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcccaagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 25 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 26 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatccccaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 27 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISPIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 28 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagccccatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 29 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 30 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaaccctgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 31 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTPKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 32 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcacccccaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 33 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLPGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 34 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctgcctggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 35 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKPSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 36 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcctagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 37 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSPPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 38 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctggagggcgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagccctcccaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

SEQ ID NO: 39 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYMNAVTELQLLMQSTPATNNRARRELPRFMNYTLNNAKKTNVTLSKKRKRRFLGFLLGVGSAIASGVAVSKVLHLPPEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYCCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASICQVNEKINQSLAFIRKSDELLHNVNAGKSTTNIMITTIIIVIIVILLSLIAVGLLLYCKARSTPVTLSKDQLSGINNIAFSN

SEQ ID NO: 40 - DNA, 1725 bp, synthetic construct
  source 1..1725: mol_type = other DNA
atggagctgctgatcctgaaggccaatgccatcaccacaatcctgaccgccgtgaccttctgtttcgccagcggccagaacatcaccgaggagttctaccaaagcacctgttctgccgtgagcaagggatacctgagcgctctgagaaccggctggtacaccagcgtgatcaccatcgagctgagcaacatcaagaagaacaagtgcaacggcaccgacgccaaggtgaagctgatcaagcaggagctggacaagtacatgaacgccgtgaccgagctgcagctgctgatgcagtctacacaggccaccaacaacagagccaggagagagctgccccggttcatgaactacaccctgaacaacgccaagaagaccaacgtgaccctgagcaagaagaggaagagacggtttctgggattcctgctgggagtgggatctgctatcgccagcggagtggccgtgagcaaagtgctgcacctgcctcccgaagtgaacaagatcaagagcgccctgctgagcaccaacaaagccgtggtttctctgagcaatggcgtgagcgtgctgaccagcaaggtgctggacctgaagaactacatcgacaagcagctgctgcccatcgtgaacaagcagagctgcagcatcagcaacatcgagaccgtgatcgagttccagcagaagaacaaccggctgctggagatcacccgggaatttagcgtgaacgccggcgtgaccacacccgtgagcacatacatgctgacaaactctgagctgctgagcctgatcaacgacatgcccatcaccaacgaccagaagaagctgatgagcaacaacgtgcagatcgtgcggcagcagagctacagcatcatgagcatcatcaaggaggaagtgctggcctacgtggtgcagctgcctctgtatggagtgatcgataccccatgttggaagctgcacaccagccctctgtgcaccaccaacacaaaggagggcagcaacatctgcctgaccagaaccgacagaggatggtactgcgacaatgccggcagcgttagctttttcccccaggccgagacctgcaaggtgcagagcaacagagtgttctgcgataccatgaacagcctgaccctgcccagcgaggtgaacctgtgcaacgtggacatcttcaaccccaagtactgttgcaagatcatgaccagcaagaccgacgtgtccagcagcgtgatcacaagcctgggcgccatcgtgagctgttatggcaagaccaagtgcaccgccagcaacaagaaccggggcatcatcaagaccttcagcaacggctgcgactatgtgagcaacaagggcgtggacaccgtgagcgtgggcaacaccctgtactacgtgaacaagcaggagggcaagagcctgtacgtgaagggcgagcccatcatcaacttctacgacccactggtgttcccaagcgacgagttcgacgccagcatctgtcaggtgaacgagaagatcaaccagagcctggccttcattcgcaagagcgacgagctgctgcataacgtgaatgccggcaagagcaccaccaacatcatgatcaccaccatcattatcgtgatcatcgtgatcctgctgagcctgatcgccgtgggcctcctgctgtattgtaaggccagaagcacccccgtgacactgagcaaggaccagctgtctggcatcaacaacatcgccttcagcaactga

(19) United States
(12) Patent Application Publication
(10) Pub. No.: US 2026/0347412 A1
(43) Pub. Date: Nov. 5, 2026

(54) MUTANT OF RSV PRE-FUSION CONFORMATION F PROTEIN AND USE THEREOF

(71) Applicants: BEIJING NEOCURNA BIOTECHNOLOGY CORPORATION, Beijing (CN); SHENZHEN NEOCURNA BIOTECHNOLOGY CORPORATION, Guangdong (CN); NEOCURA BIO-MEDICAL TECHNOLOGY CO.,LTD, Guangdong (CN)

(72) Inventors: Yi WANG, Beijing (CN); Shuai BI, Beijing (CN); Wei KANG, Beijing (CN); Dandan LI, Beijing (CN); Ji WAN, Beijing (CN); Youdong PAN, Beijing (CN); Zhao ZHAO, Beijing (CN)

(73) Assignees: BEIJING NEOCURNA BIOTECHNOLOGY CORPORATION, Beijing (CN); SHENZHEN NEOCURNA BIOTECHNOLOGY CORPORATION, Guangdong (CN); NEOCURA BIO-MEDICAL TECHNOLOGY CO.,LTD, Guangdong (CN)

(21) Appl. No.: 19/464,619

(22) Filed: Jan. 30, 2026

Related U.S. Application Data
(63) Continuation of application No. PCT/CN2024/110284, filed on Aug. 7, 2024.

(30) Foreign Application Priority Data
Aug. 7, 2023 (CN) 202310985912.8
Jul. 16, 2024 (CN) 202410953045.4

(51) Int. Cl. C07K 14/005; A61K 39/00; A61K 39/155

(52) U.S. Cl. CPC C07K 14/005; A61K 39/155; A61K 2039/53; C12N 2760/18522; C12N 2760/18534

(57) ABSTRACT

Provided is a mutant of a respiratory syncytial virus (RSV) pre-fusion conformation F protein. The mutant includes an amino acid mutation relative to the amino acid sequence of a wild-type RSV F protein, the amino acid mutation being amino acids at a position 392 and a position 493 or a position near the position 493 being substituted by C, and a disulfide bond being formed between a position 392C and a position 493C or a position near the position 493C after the mutation. Also provided are a related DNA coding sequence, recombinant plasmid and mRNA sequence and a corresponding RSV vaccine. The mutant is also applied to preparation of an RSV vaccine and RSV infection detection.

### CROSS-REFERENCES TO RELATED APPLICATIONS

This application is a continuation of PCT application No. PCT/CN2024/110284, filed on Aug. 7, 2024, which claims priority right of Chinese patent application No. 202310985912.8 filed on Aug. 7, 2023, entitled “Mutant of RSV Pre-fusion Conformation F Protein and Use Thereof”, and Chinese patent application No. 202410953045.4 filed on Jul. 16, 2024, entitled “Mutant of RSV Pre-fusion Conformation F Protein and Use Thereof”. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jan. 23, 2026, is named IPGD25US018P1-SEQ and is 81,645 bytes in size.

### BACKGROUND

### Technical Field

The present disclosure relates to the field of biomedical technology, particularly relates to a mutant of a respiratory syncytial virus pre-fusion conformation F protein, and an application thereof in the development of respiratory syncytial virus vaccines and diagnosis of respiratory syncytial virus infections.

### Description of Related Art

Respiratory syncytial virus (RSV) infection causes significant morbidity and mortality in infants and the elderly, with an estimated 64 million infections and 160,000 deaths worldwide every year. Pneumonia caused by RSV infection is the primary cause of clinical deaths. So far, the passive prophylaxis with monoclonal antibodies has been limited to high-risk infants due to their moderate efficacy. Currently, there is no safe and effective vaccine approved for RSV infection in China, highlighting the urgent need for RSV vaccines to provide protective immune responses.

The RSV genome is approximately 15Kb in length and contains 10 genes encoding 11 proteins, including 8 structural proteins (F, G, M2-1, M2-2, SH, N, P, L) and 3 non-structural proteins (NS1, NS2, NS3). Wherein, fusion glycoprotein (F) and attachment glycoprotein (G) are the two main envelope glycoproteins. F protein belongs to type I virus fusion protein, which mediates the fusion of virus with host cell membrane, causing the viral envelope to fuse with the host cell membrane to form a syncytium. Neutralizing antibodies targeting F protein can effectively inhibit RSV virus infection, and neutralizing antibodies induced by natural RSV infection in the human body mainly target F protein; the amino acid sequence of F protein has over 93% consistency among RSV subtypes, exhibiting far greater genetic stability than the G protein. In addition, F protein is an important target for inducing the production of CD8+T-cells in the body. Therefore, RSV F protein is an important target for the development of RSV vaccines.

The F protein of RSV has a trimeric structure that mediates the fusion of the viral envelope and host cells through conformational changes. Before fusion initiation, F protein forms a pre-fusion conformation, which is unstable and has a lower energy barrier. During RSV virus infection of host cells, the F protein undergoes a conformational transition from the pre-fusion conformation to the post-fusion conformation, mediating the fusion of the virus with the host cell membrane. The F protein in the post-fusion conformation is highly stable. The structure of F protein in the pre-fusion conformation is significantly different from that in the post-fusion conformation, with different antigenic epitopes. The pre-fusion conformation of F protein contains key neutralizing epitopes against RSV infection. When F protein transitions to the post-fusion conformation, most of the key neutralizing epitopes are lost. Therefore, maintaining the stability of the pre-fusion trimeric conformation of the F protein is crucial.

In order to solve the problem of the instability of the structure of the pre-fusion conformation F protein (pre-F), mutations are introduced at specific amino acid sites in the protein to stabilize its structure, increasing stability while preserving the neutralizing sensitive epitopes of the pre-F conformation.

### SUMMARY

Based on this, the purpose of the present disclosure is to provide a mutant of RSV pre-fusion conformation F protein and application thereof, wherein the mutant of pre-fusion conformation F protein can improve the expression level and stability of pre-fusion trimerized conformation F protein.

The first aspect of the present disclosure provides a mutant of a respiratory syncytial virus (RSV) pre-fusion conformation F protein, wherein the mutant includes at least one amino acid mutation relative to the amino acid sequence of a wild-type RSV F protein, the amino acid mutations include amino acids at positions 392 and 493, or at position 392 and position near 493, being substituted by cysteine respectively; a disulfide bond is formed between the mutated 392C and mutated 493C, or between the mutated 392C and the mutated positions near 493.

In some embodiments, the amino acid mutations include amino acids at a position 392 and a position 493. In some embodiments, the mutation is the combination of 392C and 493C.

In some embodiments, the position near position 493 includes position 491, and the mutation is 491C.

In some embodiments, the amino acid sequence of the wild-type RSV F protein is shown in SEQ ID NO: 1.

In some embodiments, the mutant further includes at least one cavity-filling mutation, which is selected from at least one of the following position: E60I, E60Y, S62V, K80M, K87M, K87Y, V90I, S150L, S150V, S150F, S150W, V154L, K168Y, K168L, K191V, K191L, K191M, K427Y, D448V, D448Y, S190F and V207L, E60I and K191V, S150L and K191V, K191V and D448V, V154L and K191V, K168Y and K191V, K191V and K427Y, S150L and V154L, K427Y and D448V, E60I and S62V, S190F and V207L, or the above-mentioned mutation sites are replaced by any hydrophobic amino acid.

Preferably, the cavity-filling mutation is selected from at least one of the following position: K80M, K87M, K87Y, V90I, V154L, K427Y, D448Y, S190F and V207L.

In some embodiments, the mutant includes a disulfide bond formed by a mutation between a mutated position D392C and S493C, and at least one cavity-filling mutation, wherein the cavity-filling mutation is selected from the following: K80M, K87M, K87Y, V154L, D448Y, S190F, and V207L.

In some embodiments, the mutant includes the combination of D392C, S493C, and K87M.

In some embodiments, the mutant includes a disulfide bond formed between mutated D392C and S491C, and cavity-filling mutation, wherein the cavity-filling mutation includes S190F and V207L mutations.

In some embodiments, the mutant includes the combination of D392C, S491C, S190F and V207L.

In some embodiments, the mutant further includes at least one proline mutation selected from the following: Q210P, S213P, I214P, S215P, N216P, I217P, N183P, S182P, N175P, S173P, G162P, E161P, and the combination of S213P and I214P, I214P and S215P, S215P and N216P, N216P and I217P, or E161P and G162P.

In some embodiments, the mutant includes a disulfide bond formed between a mutated position D392C and S493C after mutation, and a cavity-filling mutation at a position K87M, or the combination of S190F and V207L, and further includes at least one proline mutation selected from the following: Q210P, S213P, I214P, S215P, N216P, I217P, N183P, S182P, N175P, S173P, G162P, E161P, S213P and I214P, I214P and S215P, S215P and N216P, N216P and I217P, E161P and G162P.

In some embodiments, the mutant includes a disulfide bond formed between a mutated position D392C and S491C after mutation, and a cavity-filling mutation at a position S190F and V207L, and further includes an I214P mutation.

In some embodiments, the mutant includes the combination of D392C, S491C, S190F, V207L, and I214P.

In some embodiments, the amino acid sequence of the mutant is as shown in SEQ ID NO: 3.

In some embodiments, the amino acid sequence of the mutant is as shown in SEQ ID NO: 5.

In some embodiments, the amino acid sequence of the mutant is as shown in SEQ ID NO: 7.

In some embodiments, the amino acid sequence of the mutant is selected from any one of the following: SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39.

The second aspect of the present disclosure provides a DNA sequence encoding a mutant of RSV pre-fusion conformation F protein.

In some embodiments, the coding sequence is selected from any one of the following: SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40.

The third aspect of the present disclosure provides a recombinant plasmid, including an inserted coding sequence for a mutant encoding any of the RSV pre-fusion conformation F protein.

The fourth aspect of the present disclosure provides an mRNA sequence encoding a mutant of the RSV pre-fusion conformation F protein, which can be obtained by enzyme digestion and transcription of a recombinant plasmid containing the sequence encoding any of the aforementioned mutants.

The fifth aspect of the present disclosure provides an application of the mRNA sequence or the mutant thereof in the preparation of an RSV vaccine.

The sixth aspect of the present disclosure provides an application of the mutant in the detection of respiratory syncytial virus infection.

The seventh aspect of the present disclosure provides a respiratory syncytial virus (RSV) vaccine, including at least one nucleic acid, which has an open reading frame (ORF) encoding a mutant or immunogenic fragment of the RSV pre-fusion conformation F protein, and a pharmaceutically acceptable carrier.

Compared with the existing technologies, the present disclosure has the following beneficial effects:
1. By introducing disulfide bonds at appropriate positions of amino acids (amino acid position 392 and 493 or 491), the movement of the HRB region can be restricted, thereby disulfide bonds can stabilize both the pre-fusion conformation and trimerization conformation of F protein, and significantly inhibit the transformation of F protein into the post fusion conformation.
2. On the basis of the disulfide bond D392C-S491C, introducing single point cavity-filling mutations, or on the basis of D392C-S493C, introducing specific single point cavity-filling mutations and double point cavity-filling mutations, is more conducive to stabilizing the conformation of F protein and increasing protein expression.
3. The combination of disulfide bond with cavity-filling, electrostatic mutation, and introduction of proline mutation at specific positions described in the present disclosure can further significantly improve the expression and stability of pre-fusion conformational and trimerized conformation F protein, and significantly inhibit the transformation of F protein into the fused conformation.

### BRIEF DESCRIPTION OF THE DRAWINGS

FIG. 1: pNeoCura-Bvac plasmid profile.

FIG. 2: Binding analysis of RF-NeoC-K1 mRNA transfected cells with monoclonal antibodies, wherein A. means average fluorescence intensity of the cells binding to monoclonal antibody D25; B. means average fluorescence intensity of the cells binding to monoclonal antibody AM14; C. means average fluorescence intensity of the cells binding to monoclonal antibody 4D7; D. means ratio of the cells binding to monoclonal antibody D25; E. means average fluorescence intensity of the cells binding to monoclonal antibody AM14; F. means ratio of the cells binding to monoclonal antibody 4D7.

FIG. 3: Binding analysis of RF-NeoC-K24 and RF-NeoC-K35 mRNA transfected cells with monoclonal antibodies, wherein, A. D25 monoclonal antibody; B. AM14 monoclonal antibody; C. 4D7 monoclonal antibody.

FIG. 4: Binding analysis of RF-NeoC-K133 mRNA transfected cells with monoclonal antibodies, wherein, A. D25 monoclonal antibody; B. AM14 monoclonal antibody; C. 4D7 monoclonal antibody.

FIG. 5: The protein expression levels of the mutant containing disulfide bond mutations relatived to RSV wild-type F protein, wherein A. is the protein bound with D25 monoclonal antibody, representing the expression level of the pre-fusion conformation F protein; B. is the protein bound to the AM14 monoclonal antibody, representing the expression level of the trimeric pre-fusion F protein; C. is the protein bound to the 4D7 monoclonal antibody, representing the expression level of the post-fusion conformation F protein.

FIG. 6: The expression levels of mutant mRNA containing disulfide bond mutations and single cavity filling mutations relative to the protein encoding RSV wild-type F protein mRNA, where A. is the protein bound with D25 monoclonal antibody, representing the expression level of the pre-fusion conformation F protein; B. is the protein bound to the AM14 monoclonal antibody, representing the expression level of the trimeric pre-fusion F protein; C. is the protein bound to the 4D7 monoclonal antibody, representing the expression level of the post-fusion conformation F protein.

FIG. 7: The relative protein expression levels of mutant mRNAs containing disulfide bond mutation and two cavity-filling mutations relatived to RSV wild-type F protein, wherein A. is the protein bound with D25 monoclonal antibody, representing the expression level of the pre-fusion conformation F protein; B. is the protein bound to the AM14 monoclonal antibody, representing the expression level of the trimeric pre-fusion F protein; C. is the protein bound to the 4D7 monoclonal antibody, representing the expression level of the post-fusion conformation F protein.

FIG. 8: The protein expression levels of mutant containing disulfide bond mutation, single cavity-filling mutation, and proline mutation linking the hinge region of α-helix and α-helix or j-fold relative to the mRNA encoding RSV wild-type F protein, wherein A. is the protein bound with D25 monoclonal antibody, representing the expression level of the pre-fusion conformation F protein; B. is the protein bound to the AM14 monoclonal antibody, representing the expression level of the trimeric pre-fusion F protein; C. is the protein bound to the 4D7 monoclonal antibody, representing the expression level of the post-fusion conformation F protein.

### DESCRIPTION OF THE EMBODIMENTS

In order to facilitate understanding of the present disclosure, a more comprehensive description about the present disclosure is provided below. The present disclosure can be implemented in many different forms, and is not limited to the embodiments described herein. On the contrary, these embodiments are provided to make the understanding of the disclosure more thorough and comprehensive.

The experimental methods without specifying specific conditions in the following embodiments generally follow conventional conditions, such as those described in Sambrook et al., Molecular Cloning: A Laboratory Manual, fourth edition, 2013, or the conditions recommended by the manufacturers. The various commonly used chemical reagents used in the embodiments are all commercially available products.

Unless otherwise defined, all technical and scientific terms used herein have the same meaning as commonly understood by the skilled in the art of the present disclosure. The terms used in the description of the present disclosure are for describing specific embodiments only and are not intended to limit the present disclosure. The term “and/or” used in the present disclosure includes any and all combinations of one or more associated listed items.

### DEFINITIONS

For the convenience of understanding this technology, some terms and phrases are defined below.

Disulfide bond is a chemical bond that connects thiol groups of two different cysteine residues in different peptide chains or within the same peptide chain.

Pre-fusion: conformation before fusion.
(1) Disulfide bond: As shown in Table 1, D392C and S493C undergo a mutation, resulting in the formation of a disulfide bond between them.
(2) Cavity-filling: The two groove positions in the conformation were modified (e.g. S190F and V207L in Table 1) to be filled.

The present disclosure introduces site-directed mutations at amino acid sites in the F protein sequence of respiratory syncytial virus (RSV) to form disulfide bonds and/or cavity-filling, and/or a combination of disulfide bonds and cavity-filling, thereby significantly improves the expression of the pre-fusion conformation F protein; the pre-fusion F protein can form a stable trimer and has a stable trimerized conformation. The mutant F protein used as an immunogen or vaccine can significantly increase the induction of neutralizing antibody levels against RSV.

The sequence of F protein of RSV A2 strain was used as the basic sequence for mutant mutations in the present disclosure. However, the basic sequence for mutations in the present disclosure is not limited to RSV A2 strain, but also includes other types of RSV, such as type B and clinical strains.

As is well known, RSV has two antigenic subtypes, A and B. The amino acid sequences of the mature processed F protein of the two subtypes are approximately 93% identity. As used in the present disclosure, the amino acid position is given with reference to the sequence of RSV F protein (F protein or mutant of F protein) from A2 virus strain (SEQ ID NO: 1). As used in the present disclosure, the term amino acid at position ‘x’ of the RSV F protein therefore refers to the amino acid corresponding to the amino acid at position ‘x’ in the RSV F protein of the RSV A2 virus strain as shown in SEQ ID NO: 1. Note that in the sequence numbering system used in the present disclosure, 1 refers to the N-terminal amino acid of an immature F protein (corresponding to the first amino acid of SEQ ID NO: 1). When using an other types of RSV strain instead of A2 strain, the amino acid position of the F protein will be aligned with the F protein of SEQ ID NO: 1 by inserting gaps where necessary to align the sequence of other RSV strains with the F protein of SEQ ID NO: 1, numbered according to the F protein of the A2 strain of SEQ ID NO: 1.

Sequence alignment was performed using well-known methods in the art, such as CLUSTALW, Bioedit, or CLC workbench.

The following are embodiments of the amino acid and nucleic acid sequences of partially mutated F protein. For other amino acid and nucleic acid sequences not provided, the corresponding amino acid and nucleic acid sequences can be obtained using conventional techniques based on the mutation positions given in Table 1, referring to the wild-type sequence and the following sequences.

SEQ ID NO. 1
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASISQVNEKINQSLAFIRKSDELLHNVNAGKSTTNIMITTIIIVIIVILLSLIA
VGLLLYCKARSTPVTLSKDQLSGINNIAFSN
SEQ ID NO. 2
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCAGCAAGGTGCTGGACCTGAAGAACTACATCGACAAGCAGCTGCTGCC
CATCGTGAACAAGCAGAGCTGCAGCATCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACG
ACTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCAGCATCAGCCAGGTGAACGAGAAGATCAACCAGAGCCTG
GCCTTCATTCGCAAGAGCGACGAGCTGCTGCATAACGTGAATGCCGGCAAGAGCAC
CACCAACATCATGATCACCACCATCATTATCGTGATCATCGTGATCCTGCTGAGCCT
GATCGCCGTGGGCCTCCTGCTGTATTGTAAGGCCAGAAGCACCCCCGTGACACTGA
GCAAGGACCAGCTGTCTGGCATCAACAACATCGCCTTCAGCAACTGA
(RF-NeoC-K1)
SEQ ID NO. 3
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(dRF-NeoC-K1)
SEQ ID NO. 4
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCAGCAAGGTGCTGGACCTGAAGAACTACATCGACAAGCAGCTGCTGCC
CATCGTGAACAAGCAGAGCTGCAGCATCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GTTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCAGCATCTGTCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K24)
SEQ ID NO. 5
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTEKVLDLKNYIDKQLLPILNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K24)
SEQ ID NO. 6
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCTTCAAGGTGCTGGACCTGAAGAACTACATCGACAAGCAGCTGCTGCC
CATCCTCAACAAGCAGAGCTGCAGCCCAAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GTTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCAGCATCTGTCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K35)
SEQ ID NO: 7
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTEKVLDLKNYIDKQLLPILNKQSCSPSNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K35)
SEQ ID NO: 8
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCTTCAAGGTGCTGGACCTGAAGAACTACATCGACAAGCAGCTGCTGCC
CATCCTCAACAAGCAGAGCTGCAGCCCAAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GTTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCAGCATCTGTCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K131)
SEQ ID NO: 9
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDACISQVNEKINQSLAFIRKSDELLHNVNAGKSTTNIMITTIIIVIIVILLSLI
AVGLLLYCKARSTPVTLSKDQLSGINNIAFSN
(d RF-NeoC-K131)
SEQ ID NO: 10
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCAGCAAGGTGCTGGACCTGAAGAACTACATCGACAAGCAGCTGCTGCC
CATCGTGAACAAGCAGAGCTGCAGCATCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GCTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCTGCATCAGCCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K132)
SEQ ID NO: 11
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTFKVLDLKNYIDKQLLPILNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDACISQVNEKINQSLAFIRKSDELLHNVNAGKSTTNIMITTIIIVIIVILLSLI
AVGLLLYCKARSTPVTLSKDQLSGINNIAFSN
(d RF-NeoC-K132)
SEQ ID NO: 12
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCTTTAAGGTGCTGGACCTGAAGAACTACATCGACAAGCAGCTGCTGCC
CATCCTGAACAAGCAGAGCTGCAGCATCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GCTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCTGCATCAGCCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K133)
SEQ ID NO: 13
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTFKVLDLKNYIDKQLLPILNKQSCSPSNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDACISQVNEKINQSLAFIRKSDELLHNVNAGKSTTNIMITTIIIVIIVILLSL
IAVGLLLYCKARSTPVTLSKDQLSGINNIAFSN
(d RF-NeoC-K133)
SEQ ID NO: 14
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCTTTAAGGTGCTGGACCTGAAGAACTACATCGACAAGCAGCTGCTGCC
CATCCTGAACAAGCAGAGCTGCAGCCCCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GCTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGG
ACACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCTGCATCAGCCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K5)
SEQ ID NO: 15
MELLILKANAITTILTAVTFCFASGQNITEEFYQSTCSAVSKGYLSALRTGWYTSVITIELS
NIKKNKCNGTDAKVKLIMQELDKYKNAVTELQLLMQSTQATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K5)
SEQ ID NO: 16
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCATGCAGGAGCTGGACAAGTACAAGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K6)
SEQ ID NO: 17
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSISNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K6)
SEQ ID NO: 18
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K7)
SEQ ID NO: 19
MELLILKANAITTILTAVTFCFASGQNITEEFYQSTCSAVSKGYLSALRTGWYTSVITIELS
NIKKNKCNGTDAKVKLIKQELDKYYNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K7)
SEQ ID NO: 20
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACTACAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K21)
SEQ ID NO: 21
MELLILKANAITTILTAVTFCFASGQNITEEFYQSTCSAVSKGYLSALRTGWYTSVITIELS
NIKKNKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVYTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K21)
SEQ ID NO: 22
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACAAGAACGCCGTGACCGAGCTG
CAGCTGCTGATGCAGTCTACACAGGCCACCAACAACAGAGCCAGGAGAGAGCTGCC
CCGGTTCATGAACTACACCCTGAACAACGCCAAGAAGACCAACGTGACCCTGAGCA
AGAAGAGGAAGAGACGGTTTCTGGGATTCCTGCTGGGAGTGGGATCTGCTATCGCC
AGCGGAGTGGCCGTGAGCAAAGTGCTGCACCTGGAGGGCGAAGTGAACAAGATCA
AGAGCGCCCTGCTGAGCACCAACAAAGCCGTGGTTTCTCTGAGCAATGGCGTGAGC
GTGCTGACCAGCAAGGTGCTGGACCTGAAGAACTACATCGACAAGCAGCTGCTGCC
CATCGTGAACAAGCAGAGCTGCAGCATCAGCAACATCGAGACCGTGATCGAGTTCC
AGCAGAAGAACAACCGGCTGCTGGAGATCACCCGGGAATTTAGCGTGAACGCCGGC
GTGACCACACCCGTGAGCACATACATGCTGACAAACTCTGAGCTGCTGAGCCTGAT
CAACGACATGCCCATCACCAACGACCAGAAGAAGCTGATGAGCAACAACGTGCAG
ATCGTGCGGCAGCAGAGCTACAGCATCATGAGCATCATCAAGGAGGAAGTGCTGGC
CTACGTGGTGCAGCTGCCTCTGTATGGAGTGATCGATACCCCATGTTGGAAGCTGCA
CACCAGCCCTCTGTGCACCACCAACACAAAGGAGGGCAGCAACATCTGCCTGACCA
GAACCGACAGAGGATGGTACTGCGACAATGCCGGCAGCGTTAGCTTTTTCCCCCAG
GCCGAGACCTGCAAGGTGCAGAGCAACAGAGTGTTCTGCGATACCATGAACAGCCT
GACCCTGCCCAGCGAGGTGAACCTGTGCAACGTGGACATCTTCAACCCCAAGTACT
GTTGCAAGATCATGACCAGCAAGACCGACGTGTCCAGCAGCGTGATCACAAGCCTG
GGCGCCATCGTGAGCTGTTATGGCAAGACCAAGTGCACCGCCAGCAACAAGAACCG
GGGCATCATCAAGACCTTCAGCAACGGCTGCGACTATGTGAGCAACAAGGGCGTGT
ATACCGTGAGCGTGGGCAACACCCTGTACTACGTGAACAAGCAGGAGGGCAAGAG
CCTGTACGTGAAGGGCGAGCCCATCATCAACTTCTACGACCCACTGGTGTTCCCAAG
CGACGAGTTCGACGCCAGCATCTGTCAGGTGAACGAGAAGATCAACCAGAGCCTGG
CCTTCATTCGCAAGAGCGACGAGCTGCTGCATAACGTGAATGCCGGCAAGAGCACC
ACCAACATCATGATCACCACCATCATTATCGTGATCATCGTGATCCTGCTGAGCCTG
ATCGCCGTGGGCCTCCTGCTGTATTGTAAGGCCAGAAGCACCCCCGTGACACTGAG
CAAGGACCAGCTGTCTGGCATCAACAACATCGCCTTCAGCAACTGA
(RF-NeoC-K82)
SEQ ID NO: 23
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSPSNIETVIEFQQKNNRLLEITREFS
VNAGVTTPVSTYMLTNSELLSLINDMPITNDQKKLMSNNVQIVRQQSYSIMSIIKEEVLA
YVVQLPLYGVIDTPCWKLHTSPLCTTNTKEGSNICLTRTDRGWYCDNAGSVSFFPQAET
CKVQSNRVFCDTMNSLTLPSEVNLCNVDIFNPKYCCKIMTSKTDVSSSVITSLGAIVSCY
GKTKCTASNKNRGIIKTFSNGCDYVSNKGVDTVSVGNTLYYVNKQEGKSLYVKGEPIIN
FYDPLVFPSDEFDASICQVNEKINQSLAFIRKSDELLHNVNAGKSTTNIMITTIIIVIIVILLS
LIAVGLLLYCKARSTPVTLSKDQLSGINNIAFSN
(d RF-NeoC-K82)
SEQ ID NO: 24
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCCCAAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K83)
SEQ ID NO: 25
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSIPNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K83)
SEQ ID NO: 26
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCCCCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K84)
SEQ ID NO: 27
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSISPIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K84)
SEQ ID NO: 28
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCCCCATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K85)
SEQ ID NO: 29
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSISNPETVIEFQQKNNRLLEITREFS
VNAGVTTPVSTYMLTNSELLSLINDMPITNDQKKLMSNNVQIVRQQSYSIMSIIKEEVLA
YVVQLPLYGVIDTPCWKLHTSPLCTTNTKEGSNICLTRTDRGWYCDNAGSVSFFPQAET
CKVQSNRVFCDTMNSLTLPSEVNLCNVDIFNPKYCCKIMTSKTDVSSSVITSLGAIVSCY
GKTKCTASNKNRGIIKTFSNGCDYVSNKGVDTVSVGNTLYYVNKQEGKSLYVKGEPIIN
FYDPLVFPSDEFDASICQVNEKINQSLAFIRKSDELLHNVNAGKSTTNIMITTIIIVIIVILLS
LIAVGLLLYCKARSTPVTLSKDQLSGINNIAFSN
(d RF-NeoC-K85)
SEQ ID NO: 30
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCAACCCTGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K89)
SEQ ID NO: 31
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTPKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K89)
SEQ ID NO: 32
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCCCCAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCATCAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K93)
SEQ ID NO: 33
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLPGEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K93)
SEQ ID NO: 34
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGCCTGGCGAAGTGAACAAGATCAAG
AGCGCCCTGCTGAGCACCAACAAAGCCGTGGTTTCTCTGAGCAATGGCGTGAGCGT
GCTGACCAGCAAGGTGCTGGACCTGAAGAACTACATCGACAAGCAGCTGCTGCCCA
TCGTGAACAAGCAGAGCTGCAGCATCAGCAACATCGAGACCGTGATCGAGTTCCAG
CAGAAGAACAACCGGCTGCTGGAGATCACCCGGGAATTTAGCGTGAACGCCGGCGT
GACCACACCCGTGAGCACATACATGCTGACAAACTCTGAGCTGCTGAGCCTGATCA
ACGACATGCCCATCACCAACGACCAGAAGAAGCTGATGAGCAACAACGTGCAGATC
GTGCGGCAGCAGAGCTACAGCATCATGAGCATCATCAAGGAGGAAGTGCTGGCCTA
CGTGGTGCAGCTGCCTCTGTATGGAGTGATCGATACCCCATGTTGGAAGCTGCACAC
CAGCCCTCTGTGCACCACCAACACAAAGGAGGGCAGCAACATCTGCCTGACCAGAA
CCGACAGAGGATGGTACTGCGACAATGCCGGCAGCGTTAGCTTTTTCCCCCAGGCC
GAGACCTGCAAGGTGCAGAGCAACAGAGTGTTCTGCGATACCATGAACAGCCTGAC
CCTGCCCAGCGAGGTGAACCTGTGCAACGTGGACATCTTCAACCCCAAGTACTGTTG
CAAGATCATGACCAGCAAGACCGACGTGTCCAGCAGCGTGATCACAAGCCTGGGCG
CCATCGTGAGCTGTTATGGCAAGACCAAGTGCACCGCCAGCAACAAGAACCGGGGC
ATCATCAAGACCTTCAGCAACGGCTGCGACTATGTGAGCAACAAGGGCGTGGACAC
CGTGAGCGTGGGCAACACCCTGTACTACGTGAACAAGCAGGAGGGCAAGAGCCTGT
ACGTGAAGGGCGAGCCCATCATCAACTTCTACGACCCACTGGTGTTCCCAAGCGAC
GAGTTCGACGCCAGCATCTGTCAGGTGAACGAGAAGATCAACCAGAGCCTGGCCTT
CATTCGCAAGAGCGACGAGCTGCTGCATAACGTGAATGCCGGCAAGAGCACCACCA
ACATCATGATCACCACCATCATTATCGTGATCATCGTGATCCTGCTGAGCCTGATCG
CCGTGGGCCTCCTGCTGTATTGTAAGGCCAGAAGCACCCCCGTGACACTGAGCAAG
GACCAGCTGTCTGGCATCAACAACATCGCCTTCAGCAACTGA
(RF-NeoC-K94)
SEQ ID NO: 35
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKPSCSISNIETVIEFQQKNNRLLEITREFSV
NAGVTTPVSTYMLTNSELLSLINDMPITNDQKKLMSNNVQIVRQQSYSIMSIIKEEVLAY
VVQLPLYGVIDTPCWKLHTSPLCTTNTKEGSNICLTRTDRGWYCDNAGSVSFFPQAETC
KVQSNRVFCDTMNSLTLPSEVNLCNVDIFNPKYCCKIMTSKTDVSSSVITSLGAIVSCYG
KTKCTASNKNRGIIKTFSNGCDYVSNKGVDTVSVGNTLYYVNKQEGKSLYVKGEPIINF
YDPLVFPSDEFDASICQVNEKINQSLAFIRKSDELLHNVNAGKSTTNIMITTIIIVIIVILLSL
IAVGLLLYCKARSTPVTLSKDQLSGINNIAFSN
(d RF-NeoC-K94)
SEQ ID NO: 36
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCCTAGCTGCAGCATCAGCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K96)
SEQ ID NO: 37
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLEGEVNKIKSALLSTNKA
VVSLSNGVSVLTSKVLDLKNYIDKQLLPIVNKQSCSPPNIETVIEFQQKNNRLLEITREFS
VNAGVTTPVSTYMLTNSELLSLINDMPITNDQKKLMSNNVQIVRQQSYSIMSIIKEEVLA
YVVQLPLYGVIDTPCWKLHTSPLCTTNTKEGSNICLTRTDRGWYCDNAGSVSFFPQAET
CKVQSNRVFCDTMNSLTLPSEVNLCNVDIFNPKYCCKIMTSKTDVSSSVITSLGAIVSCY
GKTKCTASNKNRGIIKTFSNGCDYVSNKGVDTVSVGNTLYYVNKQEGKSLYVKGEPIIN
FYDPLVFPSDEFDASICQVNEKINQSLAFIRKSDELLHNVNAGKSTTNIMITTIIIVIIVILLS
LIAVGLLLYCKARSTPVTLSKDQLSGINNIAFSN
(d RF-NeoC-K96)
SEQ ID NO: 38
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGGAGGGCGAAGTGAACAAGATCAA
GAGCGCCCTGCTGAGCACCAACAAAGCCGTGGTTTCTCTGAGCAATGGCGTGAGCG
TGCTGACCAGCAAGGTGCTGGACCTGAAGAACTACATCGACAAGCAGCTGCTGCCC
ATCGTGAACAAGCAGAGCTGCAGCCCTCCCAACATCGAGACCGTGATCGAGTTCCA
GCAGAAGAACAACCGGCTGCTGGAGATCACCCGGGAATTTAGCGTGAACGCCGGCG
TGACCACACCCGTGAGCACATACATGCTGACAAACTCTGAGCTGCTGAGCCTGATC
AACGACATGCCCATCACCAACGACCAGAAGAAGCTGATGAGCAACAACGTGCAGA
TCGTGCGGCAGCAGAGCTACAGCATCATGAGCATCATCAAGGAGGAAGTGCTGGCC
TACGTGGTGCAGCTGCCTCTGTATGGAGTGATCGATACCCCATGTTGGAAGCTGCAC
ACCAGCCCTCTGTGCACCACCAACACAAAGGAGGGCAGCAACATCTGCCTGACCAG
AACCGACAGAGGATGGTACTGCGACAATGCCGGCAGCGTTAGCTTTTTCCCCCAGG
CCGAGACCTGCAAGGTGCAGAGCAACAGAGTGTTCTGCGATACCATGAACAGCCTG
ACCCTGCCCAGCGAGGTGAACCTGTGCAACGTGGACATCTTCAACCCCAAGTACTG
TTGCAAGATCATGACCAGCAAGACCGACGTGTCCAGCAGCGTGATCACAAGCCTGG
GCGCCATCGTGAGCTGTTATGGCAAGACCAAGTGCACCGCCAGCAACAAGAACCGG
GGCATCATCAAGACCTTCAGCAACGGCTGCGACTATGTGAGCAACAAGGGCGTGGA
CACCGTGAGCGTGGGCAACACCCTGTACTACGTGAACAAGCAGGAGGGCAAGAGC
CTGTACGTGAAGGGCGAGCCCATCATCAACTTCTACGACCCACTGGTGTTCCCAAGC
GACGAGTTCGACGCCAGCATCTGTCAGGTGAACGAGAAGATCAACCAGAGCCTGGC
CTTCATTCGCAAGAGCGACGAGCTGCTGCATAACGTGAATGCCGGCAAGAGCACCA
CCAACATCATGATCACCACCATCATTATCGTGATCATCGTGATCCTGCTGAGCCTGA
TCGCCGTGGGCCTCCTGCTGTATTGTAAGGCCAGAAGCACCCCCGTGACACTGAGC
AAGGACCAGCTGTCTGGCATCAACAACATCGCCTTCAGCAACTGA
(RF-NeoC-K103)
SEQ ID NO: 39
MELLILKANAITTILTAVTFCFASGQNITEEFYQSTCSAVSKGYLSALRTGWYTSVITIELS
NIKKNKCNGTDAKVKLIKQELDKYMNAVTELQLLMQSTPATNNRARRELPRFMNYTL
NNAKKTNVTLSKKRKRRFLGFLLGVGSAIASGVAVSKVLHLPPEVNKIKSALLSTNKAV
VSLSNGVSVLTSKVLDLKNYIDKQLLPIVNKQSCSISNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYCCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSDEFDASICQVNEKINQSLAFIRKSDELLHNVNAGKSTTNIMITTIIIVIIVILLSLI
AVGLLLYCKARSTPVTLSKDQLSGINNIAFSN
(d RF-NeoC-K103)
SEQ ID NO: 40
ATGGAGCTGCTGATCCTGAAGGCCAATGCCATCACCACAATCCTGACCGCCGTGAC
CTTCTGTTTCGCCAGCGGCCAGAACATCACCGAGGAGTTCTACCAAAGCACCTGTTC
TGCCGTGAGCAAGGGATACCTGAGCGCTCTGAGAACCGGCTGGTACACCAGCGTGA
TCACCATCGAGCTGAGCAACATCAAGAAGAACAAGTGCAACGGCACCGACGCCAA
GGTGAAGCTGATCAAGCAGGAGCTGGACAAGTACATGAACGCCGTGACCGAGCTGC
AGCTGCTGATGCAGTCTACACAGGCCACCAACAACAGAGCCAGGAGAGAGCTGCCC
CGGTTCATGAACTACACCCTGAACAACGCCAAGAAGACCAACGTGACCCTGAGCAA
GAAGAGGAAGAGACGGTTTCTGGGATTCCTGCTGGGAGTGGGATCTGCTATCGCCA
GCGGAGTGGCCGTGAGCAAAGTGCTGCACCTGCCTCCCGAAGTGAACAAGATCAAG
AGCGCCCTGCTGAGCACCAACAAAGCCGTGGTTTCTCTGAGCAATGGCGTGAGCGT
GCTGACCAGCAAGGTGCTGGACCTGAAGAACTACATCGACAAGCAGCTGCTGCCCA
TCGTGAACAAGCAGAGCTGCAGCATCAGCAACATCGAGACCGTGATCGAGTTCCAG
CAGAAGAACAACCGGCTGCTGGAGATCACCCGGGAATTTAGCGTGAACGCCGGCGT
GACCACACCCGTGAGCACATACATGCTGACAAACTCTGAGCTGCTGAGCCTGATCA
ACGACATGCCCATCACCAACGACCAGAAGAAGCTGATGAGCAACAACGTGCAGATC
GTGCGGCAGCAGAGCTACAGCATCATGAGCATCATCAAGGAGGAAGTGCTGGCCTA
CGTGGTGCAGCTGCCTCTGTATGGAGTGATCGATACCCCATGTTGGAAGCTGCACAC
CAGCCCTCTGTGCACCACCAACACAAAGGAGGGCAGCAACATCTGCCTGACCAGAA
CCGACAGAGGATGGTACTGCGACAATGCCGGCAGCGTTAGCTTTTTCCCCCAGGCC
GAGACCTGCAAGGTGCAGAGCAACAGAGTGTTCTGCGATACCATGAACAGCCTGAC
CCTGCCCAGCGAGGTGAACCTGTGCAACGTGGACATCTTCAACCCCAAGTACTGTTG
CAAGATCATGACCAGCAAGACCGACGTGTCCAGCAGCGTGATCACAAGCCTGGGCG
CCATCGTGAGCTGTTATGGCAAGACCAAGTGCACCGCCAGCAACAAGAACCGGGGC
ATCATCAAGACCTTCAGCAACGGCTGCGACTATGTGAGCAACAAGGGCGTGGACAC
CGTGAGCGTGGGCAACACCCTGTACTACGTGAACAAGCAGGAGGGCAAGAGCCTGT
ACGTGAAGGGCGAGCCCATCATCAACTTCTACGACCCACTGGTGTTCCCAAGCGAC
GAGTTCGACGCCAGCATCTGTCAGGTGAACGAGAAGATCAACCAGAGCCTGGCCTT
CATTCGCAAGAGCGACGAGCTGCTGCATAACGTGAATGCCGGCAAGAGCACCACCA
ACATCATGATCACCACCATCATTATCGTGATCATCGTGATCCTGCTGAGCCTGATCG
CCGTGGGCCTCCTGCTGTATTGTAAGGCCAGAAGCACCCCCGTGACACTGAGCAAG
GACCAGCTGTCTGGCATCAACAACATCGCCTTCAGCAACTGA

Further detailed explanation of the present disclosure is provided below with reference to specific embodiments.

### Embodiment 1. Preparation of the Plasmid Template Encoding Wild-Type and Mutant RSV F Protein

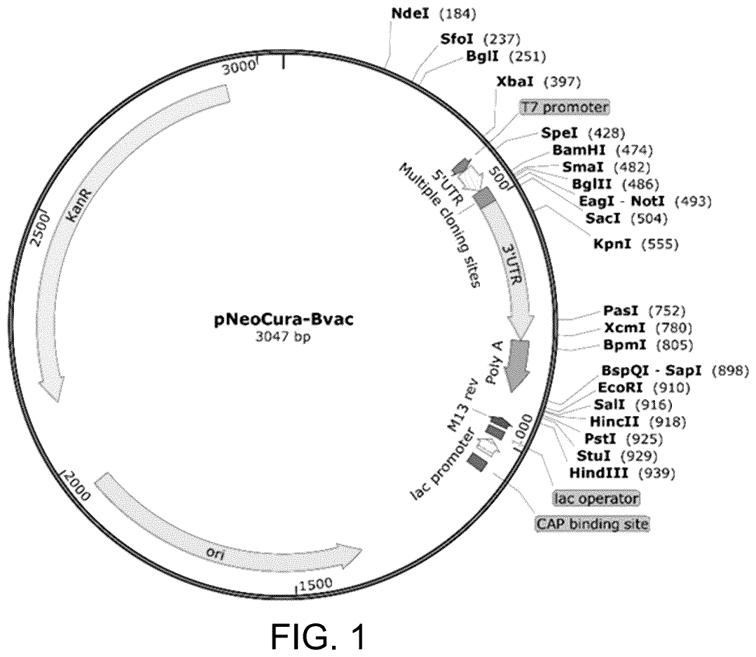

Based on the wild-type F protein sequence of RSV A2 strain (as shown in SEQ ID NO: 1) and the optimized DNA sequence encoding the wild-type F protein sequence of RSV A2 (as shown in SEQ ID NO: 2), the DNA sequence of the wild-type F protein sequence was recombined between the BamH I and Sac I sites of the pNeoCura-Bvac plasmid vector by gene synthesis to obtain the recombinant plasmid pNeoCura-Bvac-WT, which was used as a plasmid template for preparing wild-type F protein mRNA (mWT). FIG. 1 shows the plasmid profile of pNeoCura-Bvac.

According to the amino acid sequence corresponding sites of the mutant shown in Table 1, site-directed mutations were performed on the recombinant plasmid pNeoCura-Bvac-WT to obtain the corresponding recombinant plasmid pNeoCura-Bvac-RF-NoC-K1 to pNeoCura-Bvac-RF-NoC-K133 (DNA sequence: dRF-NeoC-K1 to dRF-NeoC-K133). We also constructed a recombinant plasmid pNeoCura-Bvac-Ds-Cav1, which encoded the same amino acid mutation site as Ds-Cav1, but retains the amino acid sequence of the original transmembrane region (TM region) and cytoplasmic region (CT region) of F1 at the C-terminus.

TABLE 1

| No. Of the | Disulfide bond | | |
|---|---|---|---|
| sequence | mutation | cavity-filling | Proline mutation |
| RF-NeoC-K1 | D392C-S493C | — | — |
| RF-NeoC-K2 | D392C-S493C | E60I | — |
| RF-NeoC-K3 | D392C-S493C | E60Y | — |
| RF-NeoC-K4 | D392C-S493C | S62V | — |
| RF-NeoC-K5 | D392C-S493C | K80M | — |
| RF-NeoC-K6 | D392C-S493C | K87M | — |
| RF-NeoC-K7 | D392C-S493C | K87Y | — |
| RF-NeoC-K8 | D392C-S493C | V90I | — |
| RF-NeoC-K9 | D392C-S493C | S150L | — |
| RF-NeoC-K10 | D392C-S493C | S150V | — |
| RF-NeoC-K11 | D392C-S493C | S150F | — |
| RF-NeoC-K12 | D392C-S493C | S150W | — |
| RF-NeoC-K13 | D392C-S493C | V154L | — |
| RF-NeoC-K14 | D392C-S493C | K168Y | — |
| RF-NeoC-K15 | D392C-S493C | K168L | — |
| RF-NeoC-K16 | D392C-S493C | K191V | — |
| RF-NeoC-K17 | D392C-S493C | K191L | — |
| RF-NeoC-K18 | D392C-S493C | K191M | — |
| RF-NeoC-K19 | D392C-S493C | K427Y | — |
| RF-NeoC-K20 | D392C-S493C | D448V | — |
| RF-NeoC-K21 | D392C-S493C | D448Y | — |
| RF-NeoC-K24 | D392C-S493C | S190F, V207L | — |
| RF-NeoC-K25 | D392C-S493C | E60I, K191V | — |
| RF-NeoC-K26 | D392C-S493C | S150L, K191V | — |
| RF-NeoC-K27 | D392C-S493C | K191V, D448V | — |
| RF-NeoC-K28 | D392C-S493C | V154L, K191V | — |
| RF-NeoC-K29 | D392C-S493C | K168Y, K191V | — |
| RF-NeoC-K30 | D392C-S493C | K191V, K427Y | — |
| RF-NeoC-K31 | D392C-S493C | S150L, V154L | — |
| RF-NeoC-K32 | D392C-S493C | K427Y, D448V | — |
| RF-NeoC-K33 | D392C-S493C | E60I, S62V | — |
| RF-NeoC-K35 | D392C-S493C | S190F, V207L | I214P |
| RF-NeoC-K81 | D392C-S493C | K87M | S213P |
| RF-NeoC-K82 | D392C-S493C | K87M | I214P |
| RF-NeoC-K83 | D392C-S493C | K87M | S215P |
| RF-NeoC-K84 | D392C-S493C | K87M | N216P |
| RF-NeoC-K85 | D392C-S493C | K87M | I217P |
| RF-NeoC-K86 | D392C-S493C | K87M | G184P |
| RF-NeoC-K87 | D392C-S493C | K87M | N183P |
| RF-NeoC-K88 | D392C-S493C | K87M | S182P |
| RF-NeoC-K89 | D392C-S493C | K87M | N175P |
| RF-NeoC-K90 | D392C-S493C | K87M | T174P |
| RF-NeoC-K91 | D392C-S493C | K87M | S173P |
| RF-NeoC-K92 | D392C-S493C | K87M | G162P |
| RF-NeoC-K93 | D392C-S493C | K87M | E161P |
| RF-NeoC-K94 | D392C-S493C | K87M | Q210P |
| RF-NeoC-K95 | D392C-S493C | K87M | S213P, I214P |
| RF-NeoC-K96 | D392C-S493C | K87M | I214P, S215P |
| RF-NeoC-K97 | D392C-S493C | K87M | S215P, N216P |
| RF-NeoC-K98 | D392C-S493C | K87M | N216P, I217P |
| RF-NeoC-K99 | D392C-S493C | K87M | S182P, N183P |
| RF-NeoC-K100 | D392C-S493C | K87M | N183P, G184P |
| RF-NeoC-K101 | D392C-S493C | K87M | S173P, T174P |
| RF-NeoC-K102 | D392C-S493C | K87M | T174P, N175P |
| RF-NeoC-K103 | D392C-S493C | K87M | E161P, G162P |
| RF-NeoC-K131 | D392C-S491C | / | / |
| RF-NeoC-K132 | D392C-S491C | S190F, V207L | / |
| RF-NeoC-K133 | D392C-S491C | S190F, V207L | I214P |

### Embodiment 2. Preparation of mRNAs Encoding the Wild-Type or Mutant Types of RSV F Protein

### 2.1. Preparation of Linearized Template

The recombinant plasmids were digested using the restriction endonuclease BspQ I (NEB, item number: R0712L), and the linearized plasmids were purified using VAHTS DNA Clean Beams (Novozymes, N411-01).

2.2 In Vitro Transcription to Prepare mRNA

According to the instructions of the T7 High Yield RNA Transcription Kit (N1-Me-Pseudo UTP) (Novozymes, DD4202), the linearized plasmid prepared in section 2.1 was used as a template for in vitro transcription to obtain mRNA. VAHTS RNA Clean Beads (Vazyme, N412-01) was used to purify mRNA according to the instructions.

### 2.3 Capping and Purification

The purified mRNA was caped using mRNA Cap 2′-O-Methyltransferase (Novozymes, DD4110). VAHTS RNA Clean Beads (Vazyme, item number: N412-01) was used to purify the capped mRNA to obtain the corresponding mRNA sequence.

### Embodiment 3. MRNA Transfection into Cells and Detection of F Protein Expression on Cell Surface

### 3.1 Plating of HEK293T Cells

HEK293T cells were seeded into 24 well plates (2.5×10⁵/well to 3×10⁵/well) and cultured in DMEM medium (Thermo Fisher Scientific) containing 10% fetal bovine serum and 1% penicillin streptomycin at 37 °C with 5% CO₂ for 18 hours, until reaching approximately 80% confluency prior to transfection.

3.2 mRNA Transfection into HEK293T Cells

The cell culture medium was replaced with the serum-free Opti-MEM medium (Thermo Fisher Scientific). The mRNA (0.3 g or 1 g RNA/well) was mixed with the transfection reagents Lipofectamine™ Messenger MAX™ (Thermo Fisher Scientific), then transfected into cells. After 6 hours, the cell culture medium was replaced with DMEM medium (Thermo Fisher Scientific) containing 10% fetal bovine serum and 1% penicillin streptomycin, and the cells were continued to be cultured for 24 hours.

### 3.3 Flow Cytometry Detection of the Expression of F Protein on the Surface of HEK293T Cells

After 24 hours of transfection, cells were centrifuged at 1000 g for 10 min, collected, and incubated with fluorescently labeled monoclonal antibodies. Flow cytometry (BD, FACS Celesta) was used to detect the mean fluorescence intensity (MFI) and cell proportion of F protein on the surface of cells bound to monoclonal antibodies D25, AM14, or 4D7, respectively. The monoclonal antibody D25 was prepared by the disclosed method according to (McLellan, J. S., Chen, M., Leung, S., Graepel, K. W., Du, X., Yang, Y., Zhou, T., Baxa, U., Yasuda, E., Beaumont, T., Kumar, A., Modjarrad, K., Zheng, Z., Zhao, M., Xia, N., Kwong, P. D., & Graham, B. S. (2013). Structure of RSV fusion glycoprotein trimer bound to a prefusion-specific neutralizing antibody. Science (New York, N.Y.), 340(6136), 1113-1117. https://doi.org/10.1126/science.1234914) The mean fluorescence intensity value (MFI) of D25 binding to F protein on the cell surface in was characterized as the level of F protein in the pre-fusion conformational epitope;

The monoclonal antibody AM14 was prepared by the disclosed method according to (Gilman, M. S., Moin, S. M., Mas, V., Chen, M., Patel, N. K., Kramer, K., Zhu, Q., Kabeche, S. C., Kumar, A., Palomo, C., Beaumont, T., Baxa, U., Ulbrandt, N. D., Melero, J. A., Graham, B. S., & McLellan, J. S. (2015). Characterization of a Prefusion-Specific Antibody That Recognizes a Quaternary, Cleavage-Dependent Epitope on the RSV Fusion Glycoprotein. PLoS pathogens, 11(7), e1005035.https://doi.org/10.1371/journal.ppat.1005035). The mean fluorescence intensity value (MFI) of AM14 binding to F protein on the cell surface was characterized as the level of F protein in the pre-fusion trimerized conformational epitope;

The monoclonal antibody 4D7 was prepared by the disclosed method according to (Zhang, L., Durr, E., Galli, J. D., Cosmi, S., Cejas, P. J., Luo, B., Touch, S., Parmet, P., Fridman, A., Espeseth, A. S., & Bett, A. J. (2018). Design and characterization of a fusion glycoprotein vaccine for Respiratory Syncytial Virus with improved stability. Vaccine, 36(52), 8119-8130. https://doi.org/10.1016/j.vaccine.2018.10.032). The mean fluorescence intensity value (MFI) of 4D7 binding to F protein on the cell surface in was characterized as the level of F protein in the post-fusion conformational epitope;

### Results:

(1) The second heptad repeat B(HRB) was one of the unstable regions, forming a trimeric helical stem region in the pre-fusion conformation F protein. During the transition from the pre-fusion conformation to the post-fusion conformation, it participated in the formation of three α-helices in the six helix bundle of the post-fusion conformation. The formation of disulfide bonds between amino acid 392 and amino acid 493 or 491 restricted the position movement of the HRB region, thereby stabilizing the pre-fusion conformation.

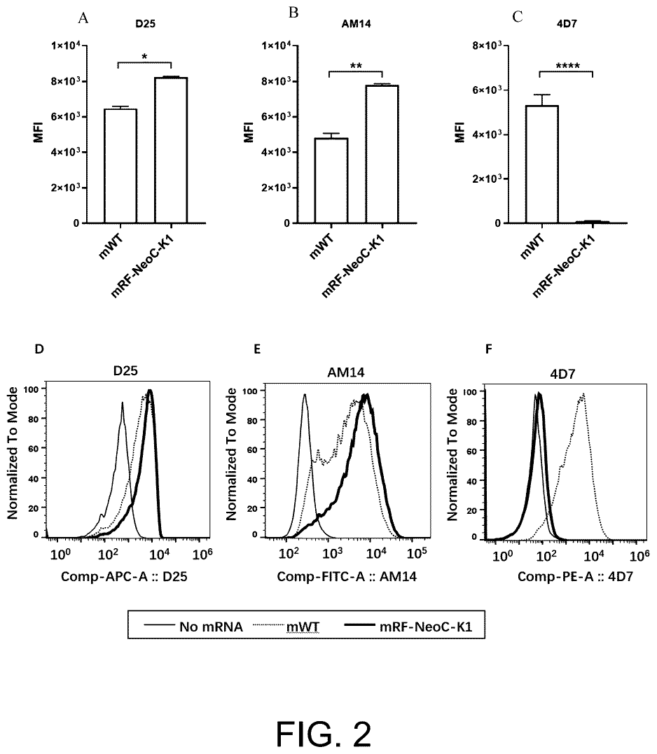

As shown in FIG. 2, compared with WT mRNA encoding wild-type F protein, the mean fluorescence intensity (MFI) and proportion of cells transfected with mRNA RF-NeoC-K1 binding to D25 were significantly increased (A and D of FIG. 2), indicating that the disulfide bond D392C-S493C mutation significantly increased the expression level and proportion of pre-fusion conformation F protein. The MFI and proportion of RF-NeoC-K1 cells transfected with mRNA binding to AM14 were significantly increased (B and E of FIG. 2), indicating that the disulfide bond D392C-S493C mutation also significantly increased the expression level and proportion of the trimeric pre-fusion F protein. In addition, the wild-type F protein encoded by WT mRNA exhibited conformational instability, resulting in high levels of MFI and proportion of binding to 4D7 in WT mRNA transfected cells, while the MFI and proportion of binding to 4D7 in cells transfected with RF-NeoC-K1 mRNA significantly decreased (C and F of FIG. 2), approaching the non-transfected mRNA group (no mRNA). The results indicated that D392C-S493C disulfide bond mutation significantly reduced the expression level and proportion of the post-fusion conformation F protein.

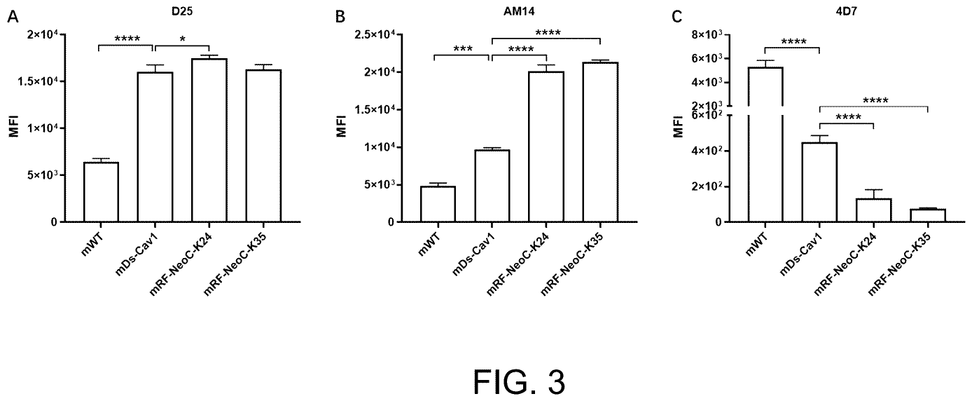

The combination of disulfide bond between D392C-S493C with cavity-filling and proline mutation at specific positions, such as cells transfected with RF-NeoC-K24 and RF-NeoC-K35, resulted in significantly higher MFI binding to D25 and AM14 compared to Ds-Cav1 (a published available stabilized pre-fusion F protein containing RSV F transmembrane domain), indicating that the levels of trimeric pre-fusion F protein encoded by mRNA RF-NeoC-K24 and RF-NeoC-K35 were significantly higher than that of Ds-Cav1 (A and B of FIG. 3); the MFI of cells transfected with RF-NeoC-K24 and RF-NeoC-K35 binding to antibody 4D7 which characterized as post-fusion conformation was significantly lower than that of Ds-Cav1, indicating that the level of post-fusion conformation F protein encoded by RF-NeoC-K24 and RF-NeoC-K35 was significantly lower than that of Ds-Cav1, and had a more stable pre-fusion conformation (C of FIG. 3).

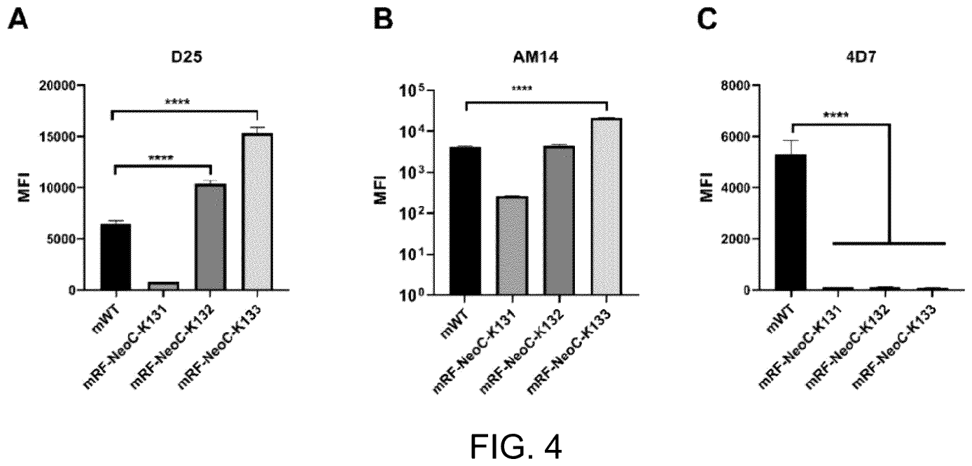

We also found that when the amino acid near position 493 (such as position 491) mutated to cysteine, a disulfide bond was formed between 392C and 491C, which helped stabilize the pre-fusion conformation of F protein, as shown in FIG. 4. Compared with the wild-type F protein(WT), the combination of disulfide bond between D392C-S491C with cavity-filling and proline mutations at specific positions, such as RF-NeoC-K133 transfected cells, significantly increased the MFI binding to D25 and AM14, indicating that the levels of trimeric pre-fusion F protein encoded by RF-NeoC-K133 was significantly higher than that of Ds-CAv1 (A and B of FIG. 4); the MFI of cells transfected with RF-NeoC-K131, RF-NeoC-K132, and RF-NeoC-K133 binding to antibody 4D7 which characterized as post-fusion conformational were significantly decreased, indicating that the level of post-fusion conformation F proteins encoded by RF-NeoC-K131, RF-NeoC-K132, and RF-NeoC-K133 had significantly reduced and had a more stable pre-fusion conformation (C of FIG. 4). Although mutation at position 392C and 491C alone did not significantly increase the MFI of cell surface F protein binding to D25 and AM14, potentially due to the overall expression level of F protein was not enhanced following RF-NeoC-K131 transfection. However, the mutation at position 392C and 491C significantly reduced the expression of the post-fusion conformation F protein, indicating that mutation 392C and 491C could effectively stabilize the F protein in trimeric pre-fusion conformation. In summary, the mutation of amino acid sites near positions 392 and 493 (such as position 491) to cysteine (C) could effectively stabilize the pre-fusion conformation F protein and inhibit the transition of post-fusion conformation F protein.

To further evaluate the effect of amino acid substitution with cysteine at positions 392 and 493 or near position 493, the effect of disulfide bond D392C-S491C was measured.

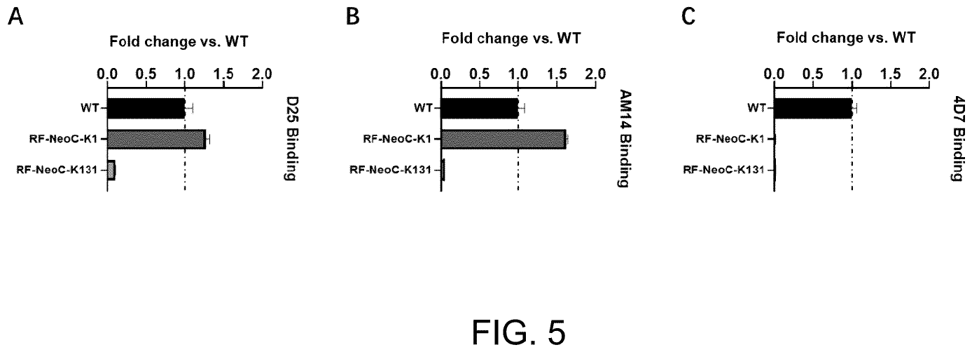

The results showed that compared with wild-type F protein encoded by WT mRNA, the MFI and proportion of cells transfected RF-NeoC-K131 mRNA binding to D25, AM14, and 4D7 were extremely low (FIG. 5); the D392C-S491C mutation significantly reduced the expression of post-fusion conformation F protein.

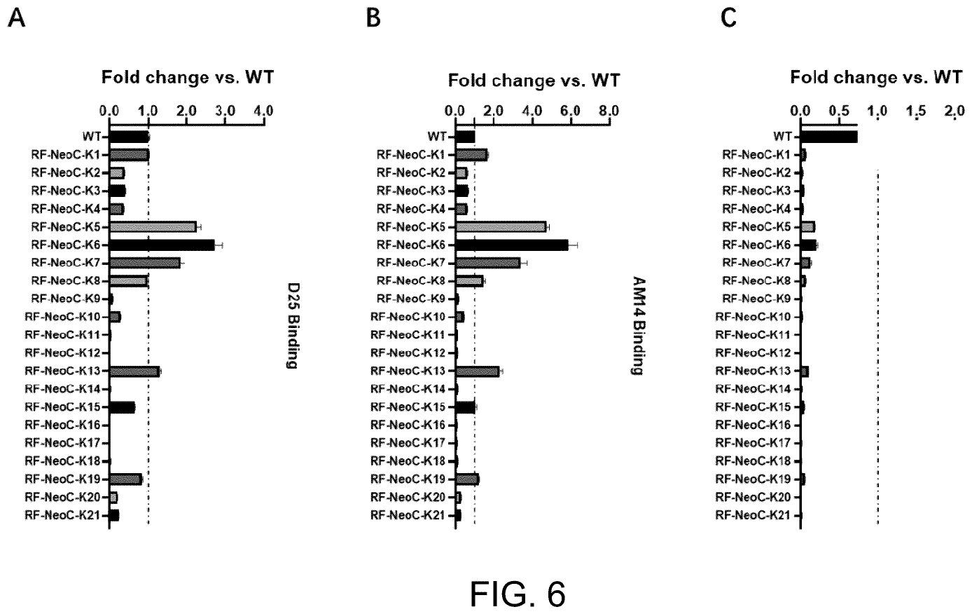

The conformation of F protein was more stabilized and the protein expression was increased after introducing hydrophobic amino acid mutations to fill the cavity. On the basis of the disulfide bond D392C-S491C, single point cavity filling mutations were introduced, as shown in FIG. 6: cavity filling mutations K80M, K87M, K87Y, V154L, K427Y further increased the expression of pre-fusion conformation F protein (A of FIG. 6) and trimeric pre-fusion F protein (B of FIG. 6), while reduced the expression of post-fusion conformation F protein (C of FIG. 6).

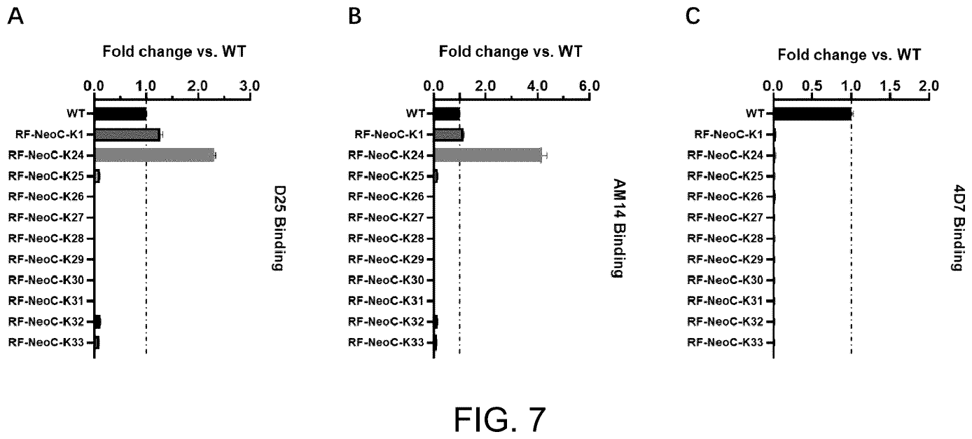

Furthermore, based on the disulfide bond D392C-S491C, two cavity-filling mutations were introduced, as shown in FIG. 7. The combination of cavity-filling mutations S190F and V207 further increased the expression of pre-fusion conformation F protein (A of FIG. 7) and trimeric pre-fusion F protein (B of FIG. 7), while reduced the expression of post-fusion conformation F protein (C of FIG. 7).

On the basis of the disulfide bond D392C-S491C, a combination of two cavity-filling mutations was introduced: E60I and K191V; S150L and K191V; K191V and D448V; V154L and K191V; K168Y and K191V; K191V and K427Y; S150L and V154L; K427Y and D448V; or E60I and S62V decreased the expression of pre-fusion conformation F protein (A of FIG. 7), trimeric pre-fusion F protein (B of FIG. 7), and post-fusion conformation F protein (C of FIG. 7).

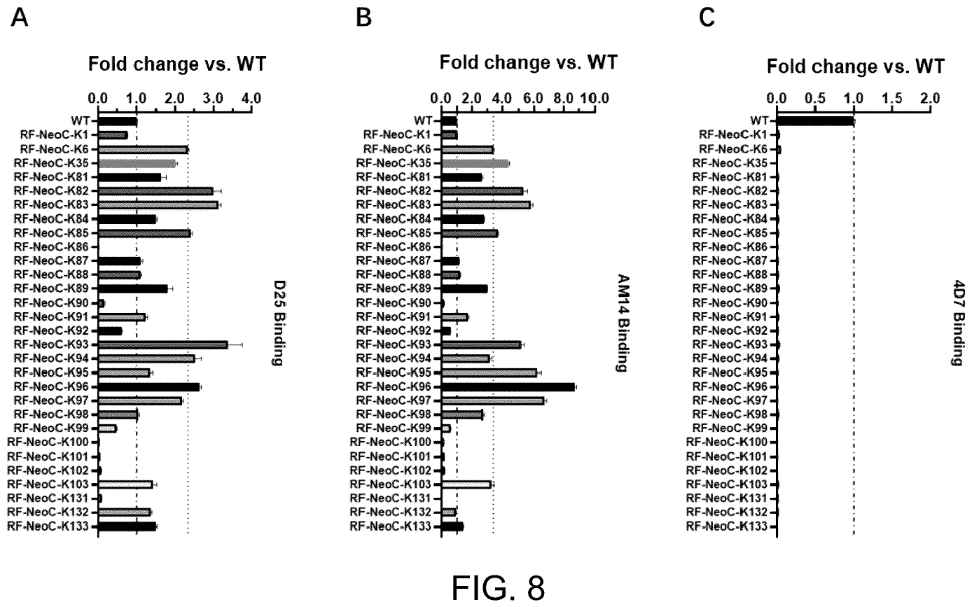

Furthermore, introducing specific proline mutations at the hinge regions linking the α-helix to the α-helix or β-sheets could stabilize the conformation of F protein and increase its expression level. Proline mutation was added at the basis of the disulfide bond D392C-S491C and cavity-filling K87M mutation (RF-NeoC-K6). The results showed that compared to the designs without proline-mutation, RF-NeoC-K6, RF-NeoC-K82 (containing I214P), RF-NeoC-K83 (containing S215P), RF-NeoC-K93 (containing E161P), and RF-NeoC-K94 (containing Q210P) all further increased the expression level of the pre-fusion conformation F protein; RF-NeoC-K82 (containing I214P), RF-NeoC-K83 (containing S215P), RF-NeoC-K85 (containing I217P), RF-NeoC-K93 (containing E161P), RF-NeoC-K95 (containing S213P, I214P), RF-NeoC-K96 (containing I214P, S215P), RF-NeoC-K97 (containing S21P, N216P) all further increased the expression level of the trimeric pre-fusion F protein; meanwhile, as shown in FIG. 8, the addition of proline mutation did not increase the expression of the post-fusion conformation F protein.

On the basis of RF-NeoC-K132 design containing disulfide bond D392C-S491C and cavity-filling mutations at position S190F and V207, proline mutation I214P was added to increase the expression of the pre-fusion conformation F protein (A of FIG. 8) and trimeric pre-fusion F protein (B of FIG. 8), while reducing the expression of the post-fusion conformation F protein (C of FIG. 8).

In summary, the disulfide bond designed in this invention, and/or its combination with cavity-filling and proline mutations at specific positions can significantly increase the expression level of trimeric pre-fusion F protein and maintain the stability of trimeric pre-fusion F protein conformation.

The embodiments above merely express several implementations of the present disclosure. The descriptions of the embodiments are relatively specific and detailed, but may not therefore be construed as the limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. A mutant of a respiratory syncytial virus (RSV) pre-fusion conformation F protein, comprising amino acid mutations relative to the amino acid sequence of a wild-type RSV F protein, wherein the amino acid mutations comprise amino acids at positions 392 and 493, or positions 392 and 491, being substituted by cysteine respectively.

2. The mutant according to claim 1, wherein the mutant further comprises at least one cavity-filling mutation, and the cavity-filling mutation is selected from at least one of the following position: E60I, E60Y, S62V, K80M, K87M, K87Y, V90I, S150L, S150V, S150F, S150W, V154L, K168Y, K168L, K191V, K191L, K191M, K427Y, D448V, D448Y, S190F and V207L, E60I and K191V, S150L and K191V, K191V and D448V, V154L and K191V, K168Y and K191V, K191V and K427Y, S150L and V154L, K427Y and D448V, E60I and S62V, S190F and V207L, or the above-mentioned mutation site is replaced by any hydrophobic amino acid.

3. The mutant according to claim 2, wherein the cavity-filling mutation is selected from at least one of the following: K80M, K87M, K87Y, V90I, V154L, K427Y, D448Y, S190F and V207L.

4. The mutant according to claim 1, wherein the mutant comprises a disulfide bond formed by a mutation between a position D392C and S493C, and at least one cavity-filling mutation, wherein the cavity-filling mutation comprises at least one selected from K80M, K87M, K87Y, V154L, D448Y, S190F, and V207L; or
the mutant comprises the formation of disulfide bonds between mutated D392C and S491C, and a cavity-filling mutation, wherein the cavity-filling mutation comprises S190F and V207L mutations.

5. The mutant according to claim 1, wherein the mutant further comprises at least one proline mutation selected from the following: Q210P, S213P, I214P, S215P, N216P, I217P, N183P, S182P, N175P, S173P, G162P, E161P, and the combination of S213P and 1214P, 1214P and S215P, S215P and N216P, N216P and I217P, or E161P and G162P.

6. The mutant according to claim 1, wherein the mutant comprises a disulfide bond formed between a mutated D392C and S493C, and a cavity-filling mutation at a position K87M, or the combination of S190F and V207L, and further comprises at least one proline mutation selected from the following: Q210P, S213P, I214P, S215P, N216P, I217P, N183P, S182P, N175P, S173P, G162P, E161P, and the combination of S213P and I214P, I214P and S215P, S215P and N216P, N216P and I217P, or E161P and G162P;
or the mutant comprises a disulfide bond formed between a mutated D392C and S491C, and a cavity-filling mutation at a position S190F and V207L, and further comprises an I214P mutation.

7. The mutant according to claim 1, wherein the mutant comprises a combination of introduced amino acid mutations selected from the group consisting of:
(1) combination of D392C, S493C, and K87M;
(2) combination of D392C, S493C, S190F, and V207L;
(3) combination of D392C, S491C, S190F, and V207L; and
(4) combination of D392C, S491C, S190F, V207L, and I214P.

8. The mutant according to claim 1, wherein the amino acid sequence of the mutant is selected from any one of the following: SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39.

9. A DNA sequence encoding a mutant of RSV pre-fusion conformation F protein according to claim 1.

10. The DNA coding sequence according to claim 9, being selected from any one of the following: SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

11. An mRNA sequence encoding a mutant of RSV pre-fusion conformation F protein according to claim 1.

12. A respiratory syncytial virus (RSV) vaccine, comprising at least one nucleic acid which has an open reading frame (ORF) encoding a mutant or immunogenic fragment of the RSV pre-fusion conformation F protein according to claim 1, and a pharmaceutically acceptable carrier.

13. The mutant according to any one of claim 2, wherein the mutant further comprises at least one proline mutation selected from the following: Q210P, S213P, I214P, S215P, N216P, I217P, N183P, S182P, N175P, S173P, G162P, E161P, and the combination of S213P and I214P, I214P and S215P, S215P and N216P, N216P and I217P, or E161P and G162P.

14. The mutant according to any one of claim 3, wherein the mutant further comprises at least one proline mutation selected from the following: Q210P, S213P, I214P, S215P, N216P, I217P, N183P, S182P, N175P, S173P, G162P, E161P, and the combination of S213P and I214P, I214P and S215P, S215P and N216P, N216P and I217P, or E161P and G162P.

15. The mutant according to any one of claim 4, wherein the mutant further comprises at least one proline mutation selected from the following: Q210P, S213P, I214P, S215P, N216P, I217P, N183P, S182P, N175P, S173P, G162P, E161P, and the combination of S213P and I214P, I214P and S215P, S215P and N216P, N216P and I217P, or E161P and G162P.

16. The mutant according to any one of claim 2, wherein the mutant comprises a disulfide bond formed between a mutated D392C and S493C, and a cavity-filling mutation at a position K87M, or the combination of S190F and V207L, and further comprises at least one proline mutation selected from the following: Q210P, S213P, I214P, S215P, N216P, I217P, N183P, S182P, N175P, S173P, G162P, E161P, and the combination of S213P and 1214P, 1214P and S215P, S215P and N216P, N216P and I217P, or E161P and G162P;
or the mutant comprises a disulfide bond formed between a mutated D392C and S491C, and a cavity-filling mutation at a position S190F and V207L, and further comprises an I214P mutation.

17. The mutant according to any one of claim 3, wherein the mutant comprises a disulfide bond formed between a mutated D392C and S493C, and a cavity-filling mutation at a position K87M, or the combination of S190F and V207L, and further comprises at least one proline mutation selected from the following: Q210P, S213P, I214P, S215P, N216P, I217P, N183P, S182P, N175P, S173P, G162P, E161P, and the combination of S213P and 1214P, 1214P and S215P, S215P and N216P, N216P and I217P, or E161P and G162P;
or the mutant comprises a disulfide bond formed between a mutated D392C and S491C, and a cavity-filling mutation at a position S190F and V207L, and further comprises an I214P mutation.

18. The mutant according to any one of claim 4, wherein the mutant comprises a disulfide bond formed between a mutated D392C and S493C, and a cavity-filling mutation at a position K87M, or the combination of S190F and V207L, and further comprises at least one proline mutation selected from the following: Q210P, S213P, I214P, S215P, N216P, I217P, N183P, S182P, N175P, S173P, G162P, E161P, and the combination of S213P and 1214P, 1214P and S215P, S215P and N216P, N216P and I217P, or E161P and G162P;
or the mutant comprises a disulfide bond formed between a mutated D392C and S491C, and a cavity-filling mutation at a position S190F and V207L, and further comprises an I214P mutation.
